(19) United States
(12) Patent Application Publication
(10) Pub. No.: US 2012/0135093 A1
(43) Pub. Date: May 31, 2012

(54) SOAP COMPOSITION FOR TREATING ACNE CONTAINING ABSOLUTE GINSENG ESSENTIAL OIL

(75) Inventors: Sang Kil Han, Daejeon (KR); Joo Yang Mang, Cheonan-si (KR)

(73) Assignee: SOONCHUNHYAND UNIVERSITY INDUSTRY ACADEMY COOPERATION FOUNDATION, Asan-si (KR)

(21) Appl. No.: 13/379,101

(22) PCT Filed: Jul. 14, 2009
(86) PCT No.: PCT/KR09/03860

§ 371 Date: Dec. 19, 2011

(51) Int. Cl. A61K 36/258; A61P 17/10; A61P 31/04

(52) U.S. Cl. 424/728

(57) ABSTRACT

The present invention discloses a soap composition containing a ginseng absolute oil, based on the finding that a ginseng extract, particularly a ginseng absolute oil, has a potent effect on the treatment of acne. A soap composition for treating acne according to one embodiment of the present invention was used, and as a result, it could be seen that the soap composition exhibited excellent effects of removing sebum and killing acne bacteria to prevent proliferation of the bacteria and infection with the bacteria, thus exhibiting a potent effect on the treatment of acne.

### TECHNICAL FIELD

The present invention relates to a soap composition for treating acne containing a ginseng absolute oil.

### BACKGROUND ART

Generally, acne is an inflammation of hair follicles on the face, particularly the cheek and the forehead, and often occurs in adolescent men and women. Acne occurs when the secretion of hormones such as androgens increases. Androgens enlarge and stimulate the sebaceous glands, causing them to produce more sebum. Acne arises when the sebaceous glands become clogged due to hyperkeratinization of hair follicles. When the sebaceous glands become clogged, the sebum is accumulated below the skin, and the hair follicle blows up like a balloon and becomes a visible comedo. In the comedo, acne bacteria (Propionibacterium acnes) that infect the sebum grow. The inflammation caused by Propionibacterium acnes can cause damage to the skin and secondary bacterial infection. In this case, the irritative components of general soap enhance initial inflammation and secondary inflammation, thus causing damage to the skin.

### DISCLOSURE

### Technical Problem

Accordingly, the present inventor has conducted systematic studies and experiments on various natural materials in order to develop a material having an acne treatment effect and, as a result, has found that a ginseng extract, particularly a ginseng absolute oil, has a potent effect on the treatment of acne, particularly an bactericidal effect against acne bacteria (Propionibacterium acnes), thereby completing the present invention. Therefore, it is an object of the present invention to provide a soap composition containing a ginseng absolute oil, which exhibits excellent effects of removing sebum and killing acne bacteria to prevent proliferation of the bacteria and infection with the bacteria, thus exhibiting a potent effect on the treatment of acne.

### Technical Solution

The present invention provides a soap composition containing a ginseng absolute oil.

### Advantageous Effects

A soap composition for treating acne according to one embodiment of the present invention was used, and as a result, it could be seen that the soap composition exhibited excellent effects of removing sebum and killing acne bacteria to prevent proliferation of the bacteria and infection with the bacteria, thus exhibiting a potent effect on the treatment of acne.

### BRIEF DESCRIPTION OF DRAWINGS

FIG. 1 is a set of photographs showing measurement results for the minimal inhibition concentration (MIC) of a ginseng absolute oil against acne bacteria (Propionibacterium acnes).

### BEST MODE

The present invention relates to a soap composition for treating acne containing a ginseng absolute oil.

Methods for extracting essential oil include a distillation method, an extraction method, a squeezing method and the like. The distillation method is widely used because it is the simplest and most economical method, but is not suitable for materials which contain large amounts of water-soluble components or are unstable in thermal conditions. The extraction method is used to extract materials which contain large amounts of water-soluble components or are unstable in thermal conditions. Also, the squeezing method is a method of obtaining an extract by squeezing.

In organic solvent extraction methods, fragrance components are extracted using various solvents, and the kind of extract varies depending on the kind of solvent. Not only volatile components, but also non-volatile components, are extracted together. Specifically, a flower or the like is immersed in a solvent at room temperature, and the fragrance components of the flower are extracted in the solvent. Then, the solvent is removed at low temperature to obtain so-called “concrete oil”. A high-purity alcohol is added to the concrete extract to extract a fragrance component, after which the alcohol is evaporated to obtain so-called “absolute oil”. A soap composition according to one embodiment of the present invention contains a ginseng absolute oil as an active ingredient and may be prepared by a conventional method. The soap composition for treating acne according to the present invention may be formulated in various forms, including liquid, cream, paste or solid forms.

A cosmetically, pharmaceutically or dermatologically acceptable medium that may be used in one embodiment of the present invention may be any medium that is suitable for the resentation forms known to those skilled in the art, for topical, oral or parenteral administration. In particular, this medium may be an oily solution, a water-in-oil emulsion, an oil-in-water emulsion, a microemulsion, an oily gel, an anhydrous gel, or a dispersion of vesicles, microcapsules or microparticles. The composition according to one embodiment of the present invention may be administered by topical application.

The active ingredient of the present invention can be easily formulated according to any conventional method using a surfactant, an excipient, a coloring agent, a flavoring agent, a preservative, a stabilizing agent, a buffer, a suspending agent, or other conventional adjuvants.

In addition, the dosage of the active ingredient will vary depending on various factors, including the age, sex and weight of the subject to be treated, the particular disease or condition to be treated, the severity of the disease or condition, the route of administration, and the judgment of the practitioner. The determination of dosage based on such factors is within the knowledge of a person skilled in the art.

### Mode for Invention

Hereinafter, a preferred embodiment of the soap composition for treating acne according to the present invention will be described in further detail with reference to examples. However, these examples are for illustrative purposes only, and the scope of the present invention is not limited to these examples.

### Example

### Preparation of Ginseng Absolute Oil

1) Undried ginseng obtained in the Geumsan Ginseng Market, Korea, was washed clean with water, dried at room temperature, and then crushed with a grinder. 15 kg of the crushed ginseng was extracted by immersion in 45 kg of hexane at room temperature for 7 days, and the extraction process was carried out twice.

2) The immersed material was filtered through a 250-mesh filter, and then passed sequentially through a 3-μm mesh filter, a 1-μm mesh filter and a 0.5-μm mesh filter, thus completely removing the ginseng residue.

3) The filtrate was concentrated under reduced pressure at 40° C. using a rotary vacuum distillation apparatus, thus obtaining 72 g of a concrete ginseng extract.

4) 72 g of the concrete ginseng extract was dissolved in 1 liter of 95% ethanol (EtOH), and allowed to stand for about one day, and then filtered through Whatman No. 5 filter paper. The filtrate was passed sequentially through a 0.8-μm mesh filter and a 0.45-μm mesh filter, thus completely removing the residue.

5) The resulting filtrate was concentrated under reduced pressure at 50° C. using a rotary vacuum distillation apparatus, thus preparing a ginseng absolute oil.

The characteristics of the ginseng absolute oil prepared in the above Example are shown in Table 1 below.

TABLE 1

| | Characteristics | Remarks |
|---|---|---|
| Oil | Absolute | |
| Appearance | Brown fluid | |
| Fragrance | Fragrance of ginseng | |
| | bark | |
| Refractive index (nₚ²⁰) | 1,450-1,550 | |
| Content of heavy metals | ≦20 ppm | Passed |
| Content of arsenic (As) | ≦2 ppm | Passed |
| Content of | None | |
| preservative | | |
| Content of | ≦100 cfu/ml | Passed |
| microorganisms | | |
| Yield (%) | 22 g (0.146%) | 15 kg raw material |

### Preparation Example

The ginseng absolute oil prepared in the above Example was used to prepared a soap composition having the components and contents (%) shown in Table 2 below.

TABLE 2

| Components | Contents (wt %) |
|---|---|
| Stearic acid | 15-20 wt % |
| Lauric acid | 5-10 wt % |
| Myristic acid | 2-6 wt % |
| Triethanolamine | 3-8 wt % |
| Sodium hydroxide | 5-10 wt % |
| Glycerin | 8-15 wt % |
| Sugar | 8-15 wt % |
| Sorbitol | 8-12 wt % |
| Example | 0.04 wt % |
| Fragrance | 1 wt % |
| Triclosan | 0.2-0.5 wt % |
| Distilled water | To 100 |

### Test Example 1

### Test for Antibacterial Activity of Ginseng Absolute Oil Against Acne Bacteria (P. acnes)

The minimal inhibition concentration (MIC) of the ginseng absolute oil against acne bacteria (Propionibacterium acnes) was measured in a laboratory using an agar dilution method.

### Test Method

(1) BHI agar medium (37 g/L Brain Heart Infusion, and 1.5% agar) is prepared in a test tube.

(2) The test tube and the medium are sterilized.

(3) A ginseng absolute oil sample is diluted serially from 5 wt % to 0.001 wt % in the prepared medium.

(4) The dilution is poured into a Petri dish to make an agar plate.

(5) Acne bacteria (Propionibacterium acnes AlCC 6919) to be tested are streaked on the medium.

(6) The acne bacteria are cultured in an incubator for anaerobic bacterial culture at 35° C. for 72 hours or more.

(7) Whether the acne bacteria have grown is observed.

### Test Results

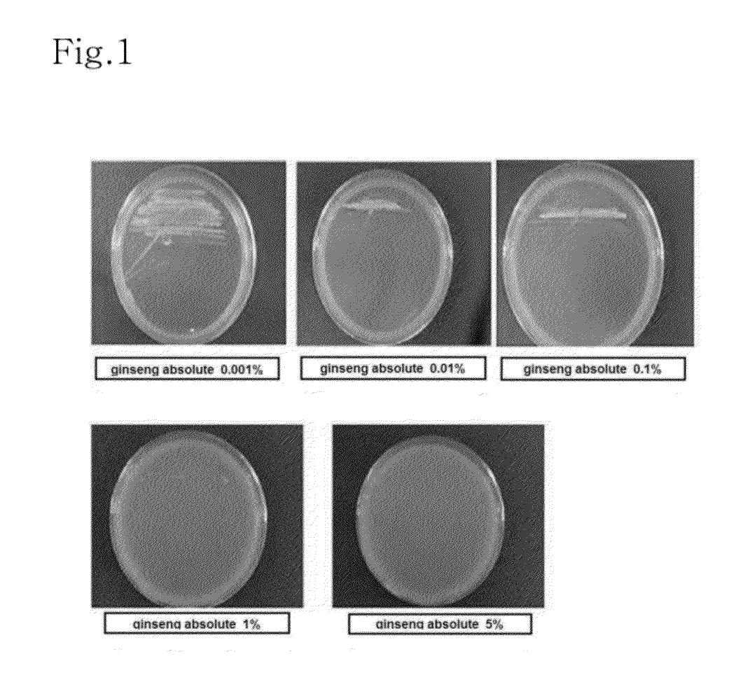

The results of measuring the minimal inhibition concentration (MIC) of the ginseng absolute oil against the acne bacteria are shown in Table 3 below and FIG. 1. As can be seen therein, the ginseng absolute oil exhibited antibacterial activity against the acne bacteria (Propionibacterium acnes) starting from a concentration of 0.01 wt %. This suggests that the ginseng absolute oil has significant antibacterial activity.

TABLE 3

| Concentration | 0.001 wt % | 0.01 wt % | 0.1 wt % | 1 wt % | 5 wt % |
|---|---|---|---|---|---|
| Growth or | Grown | Not grown | Not grown | Not | Not |
| non-growth | | | | grown | grown |

### Test Example 2

### Use Test

### Test Method

A transparent soap having the composition and contents shown in

Table 2 above was prepared and actually tested on 20 acne patients. The test was carried out for 4 weeks, and soaps other than the test soap were never used and only the test soap was used twice a day. A questionnaire was conducted each week in order to confirm the effect of the ginseng absolute oil, and each patient was photographed. The contents of the questionnaire included: “worsen”, “no changed”, “improved” and visibly improved” about inflammation, redness, and sebum secretion.

### Test Results

The effect of the test soap started to appear after 2 weeks of use, and a certain improvement was observed after 4 weeks of use. In other words, comedones significantly disappeared, and redness and the shine caused by sebum were significantly reduced.

Although the preferred embodiment of the present invention has been described for illustrative purposes, those skilled in the art will appreciate that various modifications, additions and substitutions are possible, without departing from the scope and spirit of the invention as disclosed in the accompanying claims.

1. A soap composition for treating acne containing a ginseng absolute oil.

2. The soap composition of claim 1, wherein the ginseng absolute oil is contained in an amount of 0.01-5 wt % based on a total weight of the soap composition.

3. The soap composition of claim 1, wherein the composition is in a liquid, cream, paste or solid form.

4. The soap composition of claim 1, wherein the ginseng absolute oil has a bactericidal effect against acne bacteria (Propionibacterium acnes).

5. The soap composition of claim 2, wherein the composition is in a liquid, cream, paste or solid form.

6. The soap composition of claim 2, wherein the ginseng absolute oil has a bactericidal effect against acne bacteria (Propionibacterium acnes).
